(19) United States
(12) Patent Application Publication
(10) Pub. No.: US 2013/0266487 A1
(43) Pub. Date: Oct. 10, 2013

(54) SHIELDING COLLAR

(71) Applicant: Nigel J. Osborn, Amersham (GB)

(72) Inventor: Nigel J. Osborn, Amersham (GB)

(73) Assignee: GE HEALTHCARE LIMITED, BUCKINGHAMSHIRE (GB)

(21) Appl. No.: 13/994,203

(22) PCT Filed: Dec. 16, 2011
(86) PCT No.: PCT/US11/65307

§ 371 Date: Jun. 14, 2013

Related U.S. Application Data
(60) Provisional application No. 61/424,069, filed on Dec. 17, 2010.

(51) Int. Cl. B01J 19/24; A61K 51/00

(52) U.S. Cl. CPC B01J 19/24; A61K 51/00
USPC 422/159; 422/310

(57) ABSTRACT

A shield collar (210, 410, 510) for a separations cartridge (116, 616) on a synthesis cassette may disengageably engage the separations cartridge so as to shield an operator from residual activity remaining in the separations cartridge after a synthesis operation.

### FIELD OF THE INVENTION

The present invention is directed to the field of radiopharmaceutical synthesis. More specifically, the present invention is directed to a radiation shield for a synthesis device.

### BACKGROUND OF THE INVENTION

Commercial PET production facilities are often set up solely for the production of ¹⁸F-FDG. However, as other radiotracers are developed and adopted, the production facilities will need to be able to produce these other radiotracers as well. The FASTlab® system, sold by GE Healthcare, Liege, BE, was designed from the start as a true multi-tracer platform so as to enable a given production facility to offer multiple radiotracers without requiring costly expansion of the production areas. The FAST1ab system comprises a synthesis unit which operates a single-use cassette removably mounted thereon. The spent cassette is removed after the synthesis run and replaced by a fresh cassette which may be likewise operated to perform a synthesis run. Cassettes may be tailored to produce a specific radiotracer, and the synthesis unit is programmed to operate each different type of cassette to synthesize its particular tracer.

Oftentimes several FASTlab FDG syntheses are performed by a PET center on any one day. FASTlab FDG synthesis refers to the process of producing FDG labeled with a radioisotope, including but not limited to ¹⁸F or ¹¹O, using an FDG-cassette mated to a FASTlab synthesizer. Multiple syntheses may thus be performed serially on a single synthesizer or performed using multiple synthesizers. This allows the PET center to send out multiple doses so that the patients can be scanned throughout the day and/or at different locations. It is theoretically possible to scan from a single run as much as 10 hours later for ¹⁸F-FDG, though one would probably have to use the entire batch as about 97% of the radiotracer would have decayed by then. Therefore it is much better to produce a new batch a few hours later.

Given the clinical needs to synthesize multiple radiotracers it is therefore likely that such facilities will need access to the hot cell soon after the end of one synthesis run, in order to start the synthesis run of a separate tracer. Residual activity in the cassette after a synthesis poses an exposure hazard to the operator who is replacing a spent cassette with a new one. Thus the operator is at risk of exposure to the residual activity occurs after synthesis when the operator transfers the spent cassette to a shielded disposal container. Access by an operator to the hot cells (ie, the production chambers where radioactive products are synthesized) to transfer the spent cassette will thus be restricted until the residual activity on the spent cassette below an established limit. While the FASTlab runs a rinse stage after the synthesis procedure which is designed to optimize the removal of such residual activity, given the scarcity of reagents available at this stage in the synthesis, it may be difficult to get the activity down to the levels quoted for FDG in the target product profile (<0.5% of start activity within 30 minutes from end of process), which is generally viewed as an ‘acceptable’ level of residual activity.

For instance, the present production of Fluciclatide using FASTlab leaves around 5% of the starting activity on the MCX cartridge of the cassette after the synthesis run (but prior to the rinse stage). If the synthesis is performed on a 37 GBq scale and the level of activity on the MCX cartridge can be reduced to 2.5%, however this still equates to around 433 MBq after 2 hours decay or 1678 μSv/hour at 20 cm distance. Running two back-to-back syntheses runs in quick succession (i.e., on two different cassettes), is thus technically difficult due to the residual dose to which the operator would be exposed during spent-cassette dismounting procedures. This requires more time before an operator may remove the spent cassette and then load a fresh new cassette onto the synthesizer. There is therefore a need for means to reduce operator exposure to latent radiation on a spent cassette so to better enable faster turn-around for mounting a new cassette to a synthesis device.

### BRIEF DESCRIPTION OF THE DRAWINGS

FIG. 1 depicts a fully assembled radiosynthesis cassette employing a separations cartridge.

FIG. 2 shows the numbering of each position of the cassette manifold of the cassette of FIG. 1.

FIG. 3 depicts the connections to the manifold of the cassette of FIG. 1.

FIG. 4 is a PET-CT scan depicting the residual activity within a separation cartridge after a synthesis run.

FIG. 5 is a side lavational view of a shield collar of the present invention.

FIG. 6 is a top view of the shield collar of FIG. 5.

FIG. 7 is a cross-sectional view of the shield collar of FIG. 5, taken through the line 7-7 of FIG. 6.

FIG. 8 is a partial cross-sectional view depicting the fit of a separation cartridge within the shield collar of the present invention.

FIGS. 9-11 depict a shield collar of FIG. 5 inserted over a separation cartridge of a synthesis cassette.

FIG. 12 is a bottom view of the shield collar of FIG. 5 modified to include a pair of anti-rotation ribs on its outer surface.

FIG. 13 is a top view of two-piece shield collar of the present invention.

FIG. 14 is a side lavational view of one component of the shield collar of FIG. 13.

FIG. 15 is a side lavational view of the other component of the shield collar of FIG. 13.

FIG. 16 is a top lavational view of another one piece shielding collar of the present invention.

FIG. 17-19 depict the insertion of the one-piece shield collar of FIG. 16 into a synthesis cassette of the present invention.

FIG. 20 depicts the measured residual activity remaining on a FASTlab cassette for synthesizing Flutemetamol both before and after the rinse stage.

FIG. 21 depicts the measured residual activity remaining on a FASTlab cassette for synthesizing Fluciclatide both before and after the rinse stage.

### DETAILED DESCRIPTION OF THE PREFERRED EMBODIMENT

In view of the needs of the art, the present invention provides a collar specific for a separations cartridge used on synthesis cassette that can shield the operator from residual activity on the cartridge during the short time required for the dismounting procedure. For example, the radiation-shielding collar that may be sized to fit over a solid phase extraction cartridge on a FAST1ab® cassette and thus provide significant shielding of the operator from the extraneous dose. A 1 cm thickness lead collar will reduce the dose by around 80% (347 μSv/hr for the case above), or by almost 96% (72 μSv/hr) for a 2 cm collar, and could easily be re-used once the activity has dropped to near background levels. The present invention may alternatively be formed from tungsten. Tungsten collars are relatively more effective at shielding and may be more appropriate in the tight space present. Desirably, the shielding collar is non-disposable, in that it may be removed from a cassette once the activity has decayed to near zero, or a safe level after a synthesis run and placed on a fresh cassette prior to its mounting to the synthesis device.

The shielding collars of the present invention may be placed over the separations cartridge without requiring disconnection of the cartridge from either the cassette or its associated tubing. The present invention is thus able to maintain the GMP-condition of the provided cassette. Additionally, as the shielding collar of the present invention may be removed from one cassette and emplaced on another, the unit cost of the cassette need not be negatively affected and any shipping (e.g., transportation vibration, weight) issues provided by a mounted shielding collar may be obviated. As such the shielding collar of the present invention may be provided in a kit with a synthesis cassette which includes a separations cartridge used for the synthesis of a radiotracer. The shield collar is adaptable to disengageably engage the separations cartridge so as to shield an operator from residual activity remaining in the separations cartridge after a synthesis operation.

Commercially, many PET production facilities must be able to perform several back-to-back production runs of radiotracers. By introducing a shielding collar for the SPE cartridges, an operator may now replace the spent cassette sooner, as the effective dose to the operator will be limited by the collar, thus shortening the turn-around time for resuming tracer production using the synthesis device. Thus, even if a prior synthesis run results in significant levels of activity on the solid-phase extraction cartridge after the end of the synthesis, the present invention allows shorter down-time between synthesis runs.

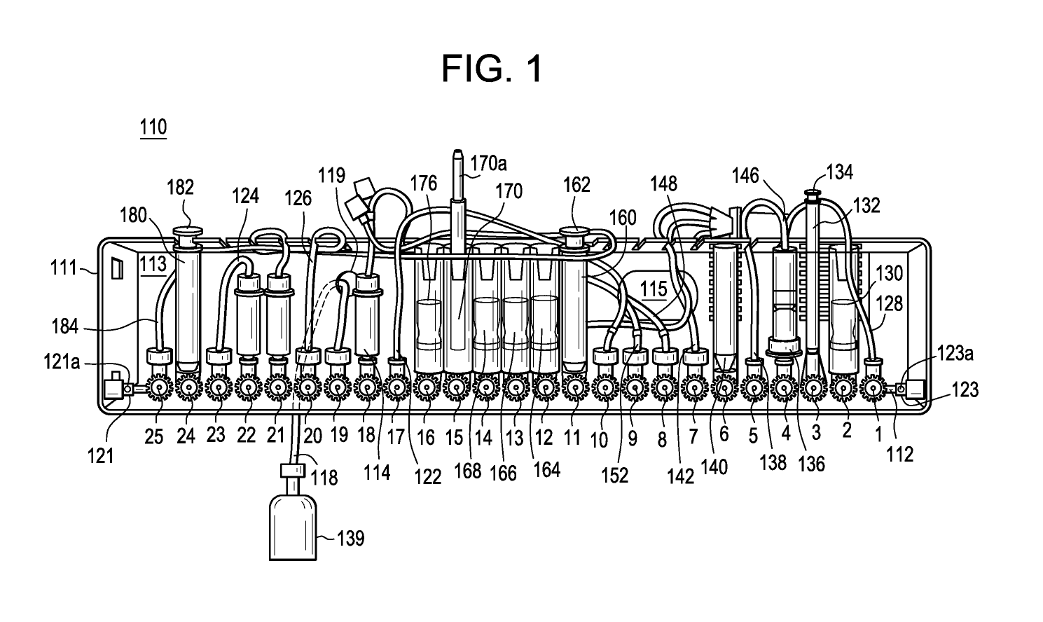

Reference is now made to FIG. 1, which depicts a disposable synthesis cassette 110 and its components. Cassette 110 is a variant of a pre-assembled unit designed to be adaptable for synthesizing clinical batches of different radiopharmaceuticals with minimal customer installation and connections. Cassette 110 includes reaction vessel, reagent vials, cartridges, filters, syringes, tubings, and connectors for synthesizing a radiotracer according to the present invention. Connections are desirably automatically made to the reagent vials by driving the septums thereof onto penetrating spikes to allow the synthesizer access to the reagents.

Cassette 110 is attachable to a synthesis device, such as FASTlab, which cooperatively engages the cassette so as to be able to actuate each of the stopcocks and syringes to drive a source fluid with a radioisotope through the cassette for performance of a chemical synthesis process. Additionally, the synthesis device can provide heat to the reaction vessel of cassette 110 as required for chemical reactions. The synthesizer is programmed to operate pumps, syringes, valves, heating element, and controls the provision of nitrogen and application of vacuum to the cassette so as to direct the source fluid into mixing with the reagents, performing the chemical reactions, through the appropriate purification cartridges, and selectively pumping the output tracer and waste fluids into appropriate vial receptacles outside the cassette. The fluid collected in the output vial is typically input into another system for either purification and/or dispensement. After product dispensement, the internal components of cassette 110 are typically flushed to remove latent radioactivity from the cassette, although some activity will remain. Cassette 110 thus can be operated to perform a two-step radiosynthesis process. By incorporating SPE cartridges on the manifold, cassette 110 is further able to provide simple purification so as to obviate the need for HPLC.

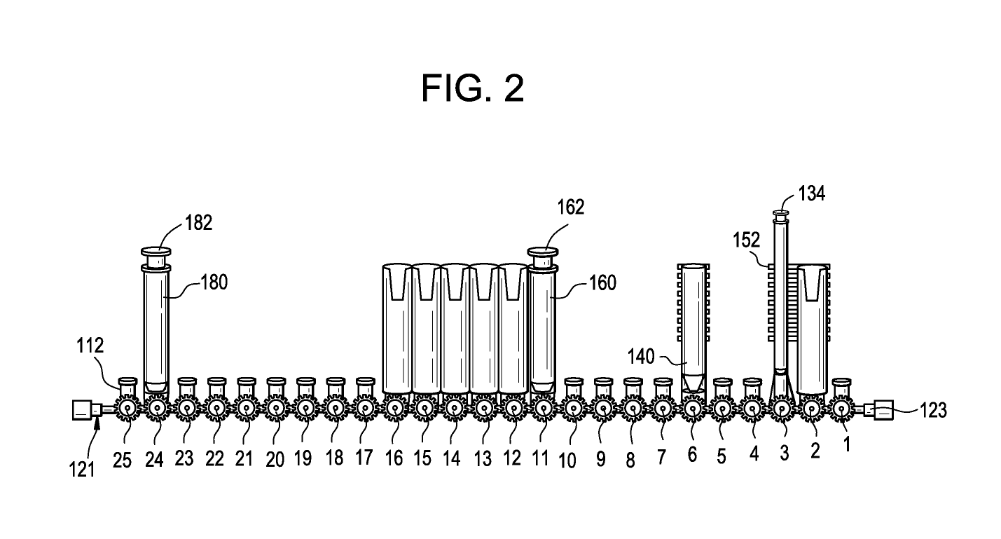

Cassette 110 includes, a manifold 112 including twenty-five 3way/3position stopcocks valves 1-25, respectively. Manifold valves 1-25 are also referred to as their manifold positions 1-25 respectively, as more clearly shown in FIG. 2. Manifold valves 1, 4-5, 7-10, 17-23, and 25 have female luer connectors projecting up therefrom. Valves 2, 6, and 12-16 have an elongate open vial housing upstanding therefrom and support an upstanding cannula therein for piercing a reagent vial inserted in the respective vial housing. Movement of the reagent vial to be pierced by the respective cannula is performed under actuation by the synthesizer device. Valves 3, 11, and 24 support an elongate open syringe barrel upstanding therefrom. Valves 1-25 include three open ports opening to adjacent manifold valves and to their respective luer connectors, cannulas, and syringe barrels. Each valve includes a rotatable stopcock which puts any two of the three associated ports in fluid communication with each other while fluidic ally isolating the third port. Manifold 112 further includes, at opposing ends thereof, first and second socket connectors 121 and 123, each defining ports 121a and 123a, respectively, for connection to a gas/vacuum source which assists in moving fluid through manifold 112. Manifold 112 and the stopcocks of valves 1-25 are desirably formed from a polymeric material, e.g. PP, PE, Polysulfone, Ultem, or Peek.

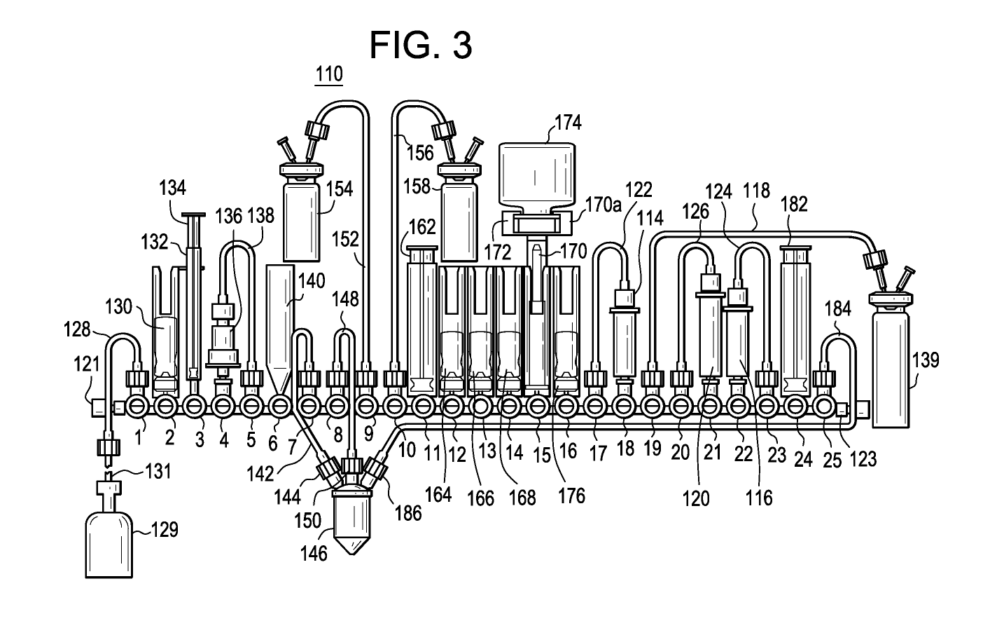

FIG. 3 depicts the connections to the manifold of cassette 110 for the production of Flutemetamol(¹⁸F) Injection, showing all tubing and prefilled reagent vials. While the cassette for producing Flutemetamol(¹⁸F) Injection is shown and described, the shield collar of the present invention is not limited to such a cassette or tracer and is contemplated to be suitable for any purification cartridge or any combination of cassette and purification cartridge for which it may be adapted. With additional reference to FIG. 1, cassette 110 includes a polymeric housing 111 having a planar major front surface 113 and defining a housing cavity 115 in which manifold 112 is supported. A first reverse phase SPE Cartridge 114 is positioned at manifold position 18 while a second reverse phase SPE cartridge 116 is positioned at manifold position 22. A normal phase (or amino) SPE cartridge 120 is located at manifold position 21. First SPE Cartridge 114 is used for primary purification. The amino cartridge 120 is used for secondary purification. The second SPE cartridge 116 is used for solvent exchange. A 50 cm to over-2 m length of Tygon tubing 118 is connected between cassette position 19 and a product collection vial 139 in which collects the formulation of the drug substance. Tubing 118 is shown in partial phantom line to indicate where is passing behind front surface 113 on the far side of manifold 112 in the view. While some of the tubings of the cassette are, or will be, identified as being made from a specific material, the present invention contemplates that the tubings employed in cassette 110 may be formed from any suitable polymer and may be of any length as required. Surface 113 of housing 111 defines an aperture 119 through which tubing 118 transits between valve 19 and the product collection vial 139. FIG. 3 depicts the same assembled manifold of the cassette and shows the connections to a vial containing a mixture of 40% MeCN and 60% water at manifold position 9, a vial of 100% MeCN at manifold position 10, a water vial connected at the spike of manifold position 14, and a product collection vial connected at manifold position 19. FIG. 3 depicts manifold 112 from the opposite face, such that the rotatable stopcocks and the ports 121a and 123a are hidden from view.

A 14 cm length of a tubing 122 extends between the free end of cartridge 114 and the luer connector of manifold valve 17. An 8 cm length of tubing 124 extends between the free end of cartridge 116 and the luer connector of manifold valve 23. A 14 cm length of tubing 126 extends between the free end of cartridge 120 and the luer connector of manifold valve 20. Additionally, tubing 128 extends from the luer connector of manifold valve 1 to a target recovery vessel 129 (shown in FIG. 3) which recovers the waste enriched water after the fluoride has been removed by the QMA cartridge. The free end of tubing 128 supports a connector 131, such as a luer fitting or an elongate needle and associated tubing, for connecting the cavity to the target recovery vessel 129. In the method of the present invention, the radioisotope is [¹⁸F]fluoride provided in solution with H₂[¹⁸O] target water and is introduced at manifold valve 6 .

A tetrabutylammonium bicarbonate eluent vial 130 is positioned within the vial housing at manifold valve 2 and is to be impaled on the spike therein. An elongate 1 mL syringe pump 132 is positioned at manifold valve 3. Syringe pump 132 includes an elongate piston rod 134 which is reciprocally moveable by the synthesis device to draw and pump fluid through manifold 112 and the attached components. QMA cartridge 136 is supported on the luer connector of manifold valve 4 and is connected via a 14 cm length of silicone tubing 138 to the luer connector of manifold position 5. Cartridge 136 is desirably a QMA light carbonate cartridge sold by Waters, a division of Millipore. The tetrabutylammonium bicarbonate in an 80% acetonitrile; 20% water (v/v) solution provides elution of [¹⁸F]fluoride from QMA and phase transfer catalyst. A fluoride inlet reservoir 140 is supported at manifold valve 6.

Manifold valve 7 supports a tubing 142 at its luer connector which extends to a first port 144 of a reaction vessel 146. The luer connector of manifold valve 8 is connected via a 14 cm length of tubing 148 to a second port 150 of reaction vessel 146. The luer connector of manifold valve 9 is connected via a 42cm length of tubing 152 to a vial 154 containing a mixture of 40% MeCN and 60% water (v/v). The acetonitrile and water mixture is used to enable primary purification of Flutemetamolat the first SPE cartridge 114. The luer connector of manifold valve 10 is connected via a 42 cm length of tubing 156 to a vial 158 containing 100% MeCN used for conditioning of the cartridges and the elution of Flutemetamolfrom the first SPE cartridge 114. Manifold valve 11 supports a barrel wall for a 5 ml syringe pump 160. Syringe pump 160 includes an elongate piston rod 162 which is reciprocally moveable by the synthesis device so as to draw and pump fluid through manifold 112. The vial housing at manifold valve 12 receives vial 164 containing 6-ethoxymethoxy-2-(4′-(N-formyl-N-methyl)amino-3′-nitro)phenylbenzothiazole). The vial housing at manifold valve 13 receives a vial 166 containing 4M hydrochloric acid. The hydrochloric acid provides deprotection of the radiolabelled intermediate. The vial housing at manifold valve 14 receives a vial 168 of a methanol solution of sodium methoxide. The vial housing at manifold valve 15 receives an elongate hollow spike extension 170 which is positioned over the cannula at manifold valve 15 and provides an elongate water bag spike 170a at the free end thereof. Spike 170 pierces a cap 172 of a water bottle 174 containing water for both diluting and rinsing the fluid flowpaths of cassette 110. The vial housing at manifold valve 16 receives a vial 176 containing ethanol. Ethanol is used for the elution of the drug substance from the second SPE cartridge 116. The luer connector of manifold valve 17 is connected to a 14 cm length of silicone tubing 122 to SPE cartridge 114 at position 18. Manifold valve 24 supports the elongate barrel of a 5 ml syringe pump 180. Syringe pump 180 includes an elongate syringe rod 182 which is reciprocally moveable by the synthesis device to draw and pump fluid through manifold 112 and the attached components. The luer connector of manifold valve 25 is connected to a 42 cm length of a tubing 184 to a third port 186 of reactor vessel 146.

Cassette 110 is mated to an automated synthesizer having rotatable arms which engage each of the stopcocks of valves 1-25 and can position each in a desired orientation throughout cassette operation. The synthesizer also includes a pair of spigots, one of each of which insert into ports 121a and 123a of connectors 121 and 123 in fluid-tight connection. The two spigots respectively provide a source of nitrogen and/or a vacuum to manifold 112 so as to assist in fluid transfer therethrough and to operate cassette 110 in accordance with the present invention. The free ends of the syringe plungers are engaged by cooperating members from the synthesizer, which will then apply the reciprocating motion thereto within the syringes. A bottle 174 containing water is fitted to the synthesizer then pressed onto spike 170 to provide access to a fluid for driving compounds under operation of the various-included syringes. The reaction vessel will be placed within the reaction well of the synthesizer and the product collection vial and waste vial are connected. The synthesizer includes a radioisotope delivery conduit which extends from a source of the radioisotope, typically either vial or the output line from a cyclotron, to a delivery plunger. The delivery plunger is moveable by the synthesizer from a first raised position allowing the cassette to be attached to the synthesizer, to a second lowered position where the plunger is inserted into the housing at manifold valve 6. The plunger provides sealed engagement with the housing at manifold valve 6 so that the vacuum applied by the synthesizer to manifold 112 will draw the radioisotope through the radioisotope delivery conduit and into manifold 112 for processing. Additionally, prior to beginning the synthesis process, arms from the synthesizer will press the reagent vials onto the cannulas of manifold 112. The synthesis process may then commence.

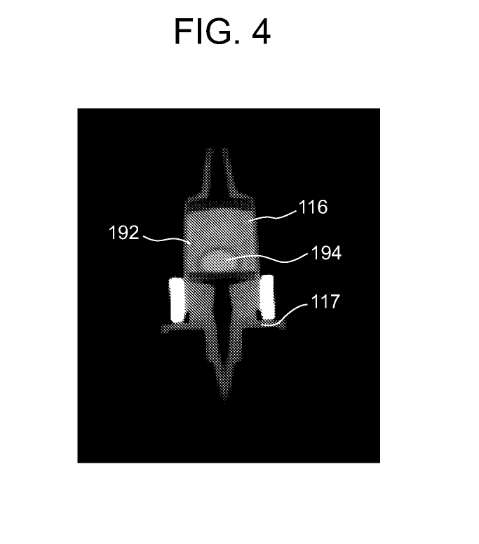

FIG. 4 shows the residual activity on a separation cartridge 116 after a synthesis run. Cartridge 116 includes a lower annular flange 117 extending thereabout. Cartridge 116 further includes an elongate tubular cartridge body 192 defining a cartridge cavity 194 therein. A separations media is supported within cartridge cavity 194. The residual activity after a synthesis run is shown to be centered within cartridge cavity 194. Additionally, post-rinsing, it has been found that most of the residual activity on cassette 110 is located in cartridge 116.

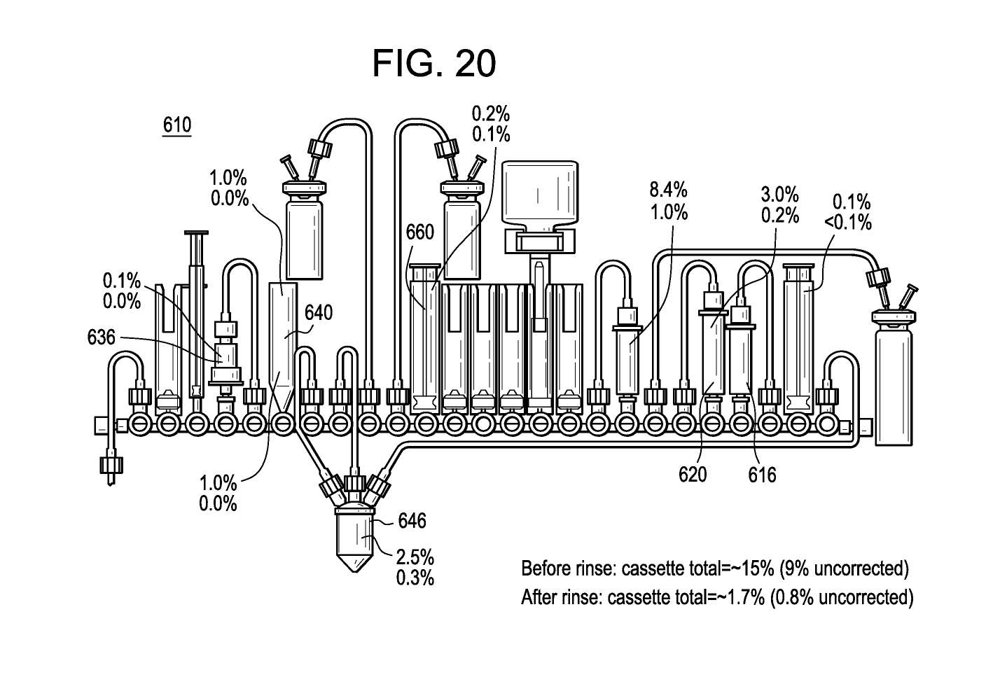

With reference to FIG. 20, a cassette 610 configured to synthesize Flutemetamol (with like numbering for like components as cassette 110 although the precise reagents and operations will be different) has been found to trap on cartridge 616 about 75% of the total residual activity on the cassette post-rinse. After synthesis, but prior to the cassette of FIG. 20 being rinsed, about 8.4% of the starting activity (that is, the total activity on QMA cartridge 636 prior to the synthesis operations in reaction chamber 646) remains trapped by cartridge 616. Additionally, about 1.0% of the starting activity is trapped on SPE Cartridge 616 and about 3.0% of the starting activity is trapped on SPE cartridge 620, about 2.5% in reaction chamber 646, 1.0% at reservoir 640, 0.1% at QMA cartridge 636, and about 0.2% at syringe pump 660. After the rinse stage, it has been found that about 1.0% of starting activity is trapped on cartridge 616, less than about 0.1% of the starting activity is trapped on SPE Cartridge 616 and about 0.2% of the starting activity is trapped on SPE cartridge 620, about 0.3% in reaction chamber 646, about 0.0% at reservoir 640, 0.0% at QMA cartridge 636, and about 0.1% at syringe pump 660. Thus, for the Flutemetamol cassette of FIG. 20, about 75% of the total residual activity residing on cassette 610 post-rinse is trapped in cartridge 616. All of the residual percentages described for FIG. 20 are corrected for decay of the radioisotope from the time on QMA cartridge 636 to the time of measurement post-synthesis and post-rinse.

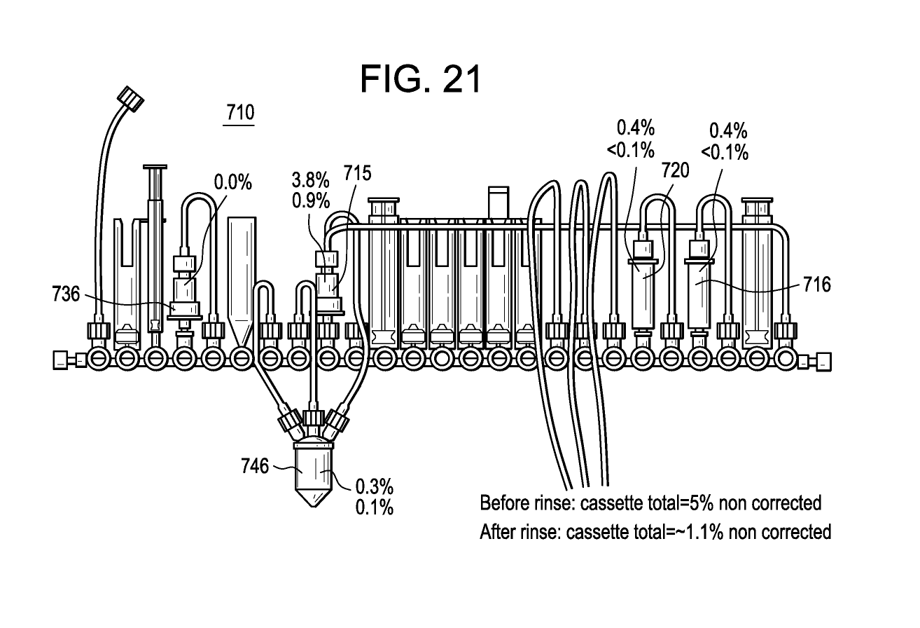

Similarly, now referring to FIG. 21, a synthesis cassette configured to synthesize Flucicilatide (with like numbering for like components as cassette 110 although the precise reagents and operations will be different) has been found to trap on cartridge 715 about 90% of the total residual activity on the cassette post-rinse. After synthesis, but prior to the cassette of FIG. 21 being rinsed, about 3.8% of the starting activity (that is, the total activity on QMA cartridge 736 prior to the synthesis operations in reaction chamber 746) remains trapped by cartridge 715. Additionally, about 0.4% of the starting activity is trapped on SPE cartridges 716 and 720 and about 0.3% in reaction chamber 746. After the rinse stage, it has been found that about 0.9% of starting activity is trapped on cartridge 715, about 0.1% is in reaction chamber 746, while both SPE cartridges 716 and 720 trap less than 0.1% of the starting activity. Thus, for the Fluciclatide cassette of FIG. 21, about 90% of the total residual activity residing on cassette 710 post-rinse is trapped in cartridge 716. It should be noted that all residual percentages described for FIG. 21 are not corrected for decay of the radioisotope from the time on QMA cartridge 736 to the time of measurement post-synthesis and post-rinse.

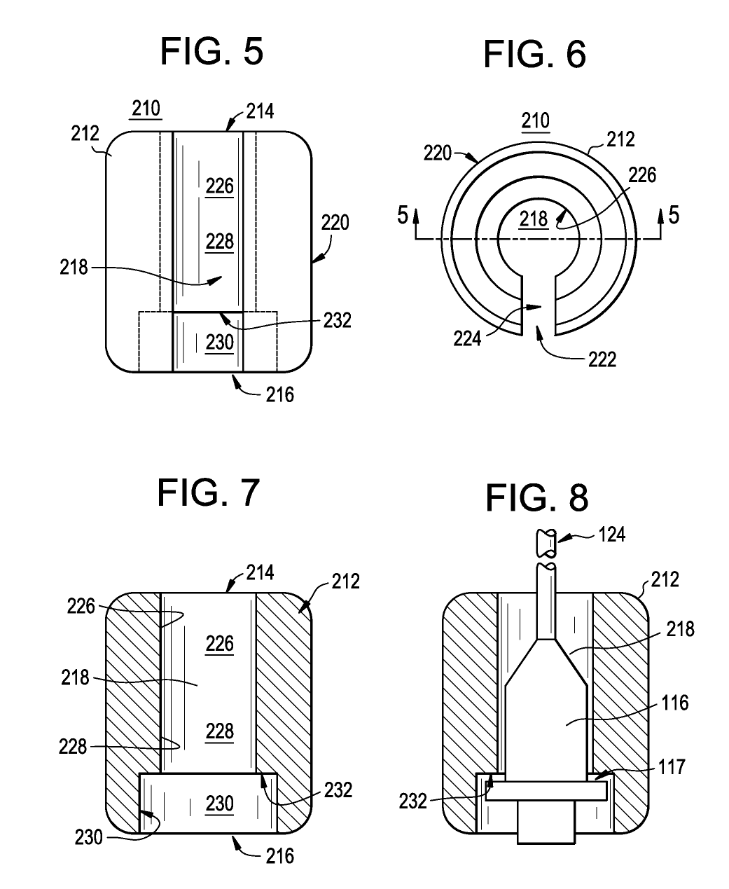

Given the localization of the residual activity in these cassettes, the present invention provides a shielding collar for the separations cartridge so as to shield an operator from the residual activity. FIGS. 5-8 depict a first shield collar 210 of the present invention. Shield collar 210 includes an collar body 212 defining opposed first and second apertures 214 and 216 and an elongate cartridge passageway 218 extending in fluid communication therebetween. Collar body 212 further includes a cylindrical outer surface 220 which further defines an elongate insertion aperture 222. Collar body 212 further defines an elongate insertion passageway 224 extending in fluid communication between cartridge passageway 218 and insertion aperture 220. Cartridge passageway 218 is further defined by an elongate cylindrical inner surface 226 of collar body 212. Surface 226 is contoured to include co-axially-aligned first cylindrical wall 228 and second cylindrical wall 230, and a transverse annular surface 232 extending between walls 228 and 230. The contour of surface 226 desirably approximates the outer surface of the separation cartridge, about which it is positioned, so as to better shield the highest activity within the cartridge. Desirably still, insertion aperture 222 and insertion passageway 224 are sized to allow the conduit extending from the cartridge 116 to pass therethrough while cartridge passageway has a larger cross-wise dimention to accommodate the cartridge body. Surface 232 desirably rests on an annular rim 117 of cartridge 116.

Collar body 212 is formed from a radiation-shielding material, such as lead, tungsten, or elkonite. Shield collar 210 is thus able to reduce an operator's exposure to the retained activity on the separation cartridge. While it is desirable that shield collar 210 sufficiently protect an operator so as to allow the operator to dismount the cassette from the synthesis unit immediately after the synthesis run, the present invention also contemplates that the protection provided by shield collar 210 allow the operator to dismount the cassette earlier than if no shielding were provided about the separation cartridge. Thus, for a given synthesis, an operator will not have to wait as long prior to dismounting the spent cassette than would be required without the present invention.

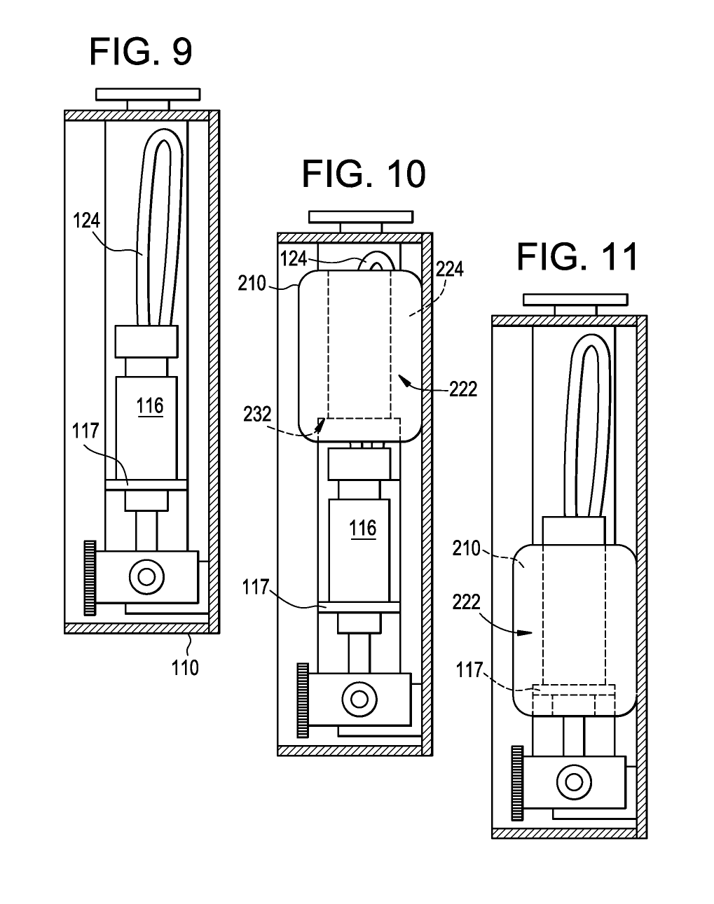

Referring now to FIGS. 9-11, to assemble collar body 212 to the separation cartridge 116 of cassette 110, an operator would pass the segment of tubing 124 ascending from cartridge 116 through insertion aperture 222 and insertion passageway 224 into cartridge passageway 218. Shield collar may then be brought down over cartridge 116 such that transverse annular surface 232 rests upon an annular flange 117 of cartridge 116. Desirably, the collar body 212 will then be rotated about cartridge 116 so that aperture 222 faces away from the planar front surface 113 of cassette 110. During cassette operation on a synthesizer, aperture 222 will face the synthesizer, thus providing shielding of radiation towards an operator. Shield collar 210 is thus able to provide shielding about cartridge 116 without requiring disconnection with either manifold 112 or ascending tubing 124, maintaining GMP-compliance of cassette 110.

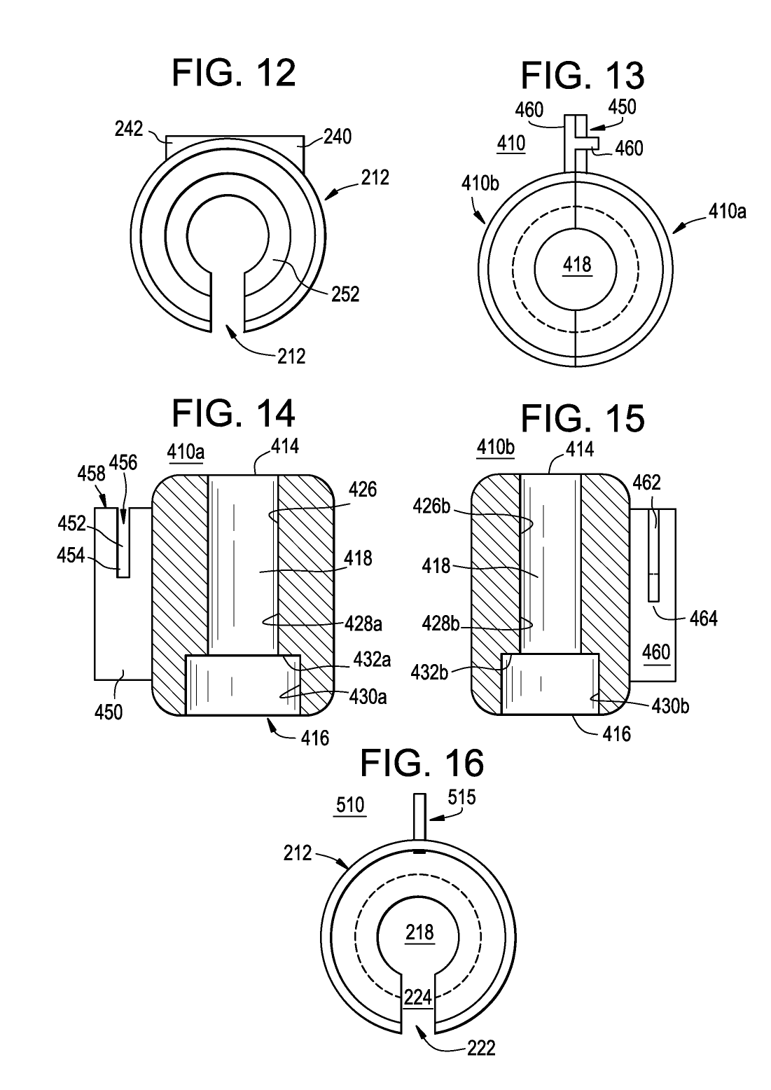

FIG. 12 is a bottom view of the shield collar 210 of FIG. 4 modified to include a pair of anti-rotation ribs, 240 and 242, on its outer surface. Ribs 240 and 242 are provided to engage planar front surface 113 of cassette 110 so as to limit rotation of the shield about the cartridge and thus maintain the orientation of aperture 222 as opening away from an operator while cassette 110 is mounted to a synthesizer.

FIGS. 13-15 depict a two-piece shield collar 410 of the present invention. Collar 410 includes mating semi-shell components 410a and 410b. Components 410a and 410b are mated to define opposed first and second apertures 414 and 416 and an elongate cartridge passageway 418 extending in fluid communication therebetween. Cartridge passageway 418 is further defined between opposed elongate cylindrical inner surface 426a and 426b of components 410a and 410b, respectively. Surfaces 426a and 426b are contoured to include first cylindrical surfaces 428a and 428b, second cylindrical surfaces 430a and 430b, and a transverse annular surface 432a and 432b extending between surfaces 428a and 430a, and 428b and 430b, respectively. The contour of surfaces 426a and 426b desirably approximates the outer surface of the separation cartridge, about which it is positioned, so as to better shield the highest activity within the cartridge.

Components 410a and 410b may be individually placed about a separations cartridge and then held together thereabout to shield the residual activity within. The present invention contemplates that a third piece may be applied to hold the two components together, such as a conventional clip, a clamp or a plastic cable tie. Desirably, components 410a and 410b include the means for holding each other together, such as mating detents or other each support a substantially planar locking flange 450 and 460, respectively. As shown in FIGS. 8-10, locking flanges 450 and 460 provide mating engagement. Flange 450 includes an elongate channel 452 having a closed end 454 and an open end 456 opening on an upper edge 458 thereof. Flange 460 supports an elongate tongue 462 which is insertable into channel 452. Tongue 462 supports a freely-extending tooth 464 such that a portion of flange 450 adjacent to closed end 454 is held between tooth 464 and flange 460.

In operation, collar component 410a is positioned to one side of a separations cartridge so that the cartridge is positioned adjacent surface 426a. Then, collar component 410b is positioned on the opposite side of the cartridge as component 410a and higher above the manifold than component 410a. The two components are brought together such that tongue 462 and tooth 464 are inserted into channel 452 so that tongue 462 is held within channel 452 and flange 450 is positioned between tooth 464 and flange 450. Shielding collar 450 may thus be held about the separations cartridge during and after a synthesis run and provide shielding to an operator while removing the cartridge (i.e., on a cassette) from the synthesizer. Shield components 410a and 410b may then be removed from about the cartridge once the residual activity within the cartridge has decayed to a safe level.

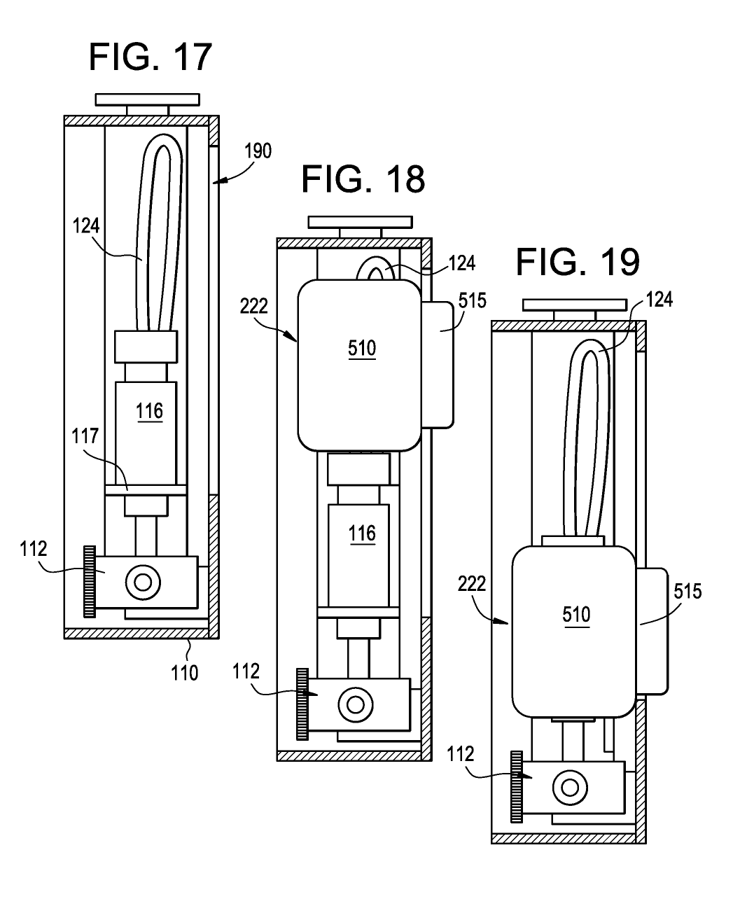

FIG. 16 is a top elevation view of another one piece shielding collar 510 of the present invention. Shielding collar 510 provides an elongate planar fin 515 to the collar body 212. Fin 515 allows easier handling of collar body 212, and is particularly suitable for applications where collar body 212 need not be rotated after emplacement over a separations cartridge. Towards this end, and with additional reference to FIGS. 17-19, the present invention further contemplates that planar front surface 113 of cassette 110 further defines an enlarged through-aperture 190 adjacent to cartridge 116. Aperture 190 is sized to allow collar 510 to be inserted over cartridge 116 such that tubing 124 is inserted through insertion aperture 222 and passageway 224 into passageway 218. Collar body 212 may then be brought straight down over cartridge 116 so that transverse surface 232 rests upon flange 117 thereof. Removal of collar 510 is contemplated to involve reversing the steps of attaching just described.

Any of the shielding collars of the present invention are contemplated to be formed from only the shielding material. Alternatively, the present invention also contemplates that the shielding collars of the present invention may additionally include a coating or elastomeric cover on one or more its surfaces. The coating or cover may, for example, be provided on just the outer surface but is desirably provided on any surface which an operator may contact while manipulating the shield.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A shield collar for a separations cartridge, said shield collar comprising:
an elongate collar body defining opposed first and second apertures and an elongate cartridge passageway extending in fluid communication therebetween, said collar body further defining an elongate insertion aperture opening on an outer surface of said collar body, said collar body further defining an elongate insertion passageway extending in fluid communication between said first aperture, second aperture, cartridge passageway and said insertion aperture.

2. A shield collar of claim 1, further comprising first and second elongate ribs extending along the outer surface of said collar body.

3. A shield collar of claim 1, wherein said collar body is formed from one of lead, tungsten, and elkonite.

4. A shield collar of claim 1, wherein said outer surface supports an elastomeric cover thereon.

5. A shield collar of claim 1, further comprising an elastomeric coating about said collar body.

6. A shield collar of claim 1, further comprising an elongate planar flange along the outer surface of said collar body.

7. A shield collar for a separations cartridge, said shield collar comprising:
first and second mateable shell components, said components defining longitudinally-opposed first and second apertures and an elongate cartridge passageway extending in fluid communication therebetween; and
a locking mechanisms for releasably joining said shell components together.

8. A shield collar of claim 7, wherein each component supports an elongate planar flange member along the outer surface thereof, wherein when said components are joined, said planar flange members are positioned adjacent one another and form the locking mechanism.

9. A cassette for synthesizing a radiotracer comprising:
an elongate manifold connected to a reaction chamber, cartridges, tubing, and at least one separations cartridge used in synthesizing the radiotracer;
a cassette housing supporting said manifold therein, said housing comprising an elongate planar base wall supporting a transversely-oriented perimetrical wall thereabout, and
a shield collar positioned about the separations cartridge so as to shield an operator from residual activity remaining therein subsequent to a synthesis operation.

10. A cassette of claim 9, wherein said planar base wall further defines a through aperture in registry with the separations cartridge through which said shield collar disengageably engages the separations cartridge.

11. A cassette of claim 10, said cassette housing further comprising a member for supporting at least some of the weight of the shield.

12. A kit for synthesizing a radiotracer comprising:
a synthesis cassette supporting at least a separations cartridge used in synthesizing the radiotracer, and
a shield collar,
wherein said shield collar is adaptable to disengageably engage the separations cartridge so as to shield an operator from residual activity remaining in the separations cartridge after a synthesis operation.
